(19) United States
(12) Patent Application Publication
(10) Pub. No.: US 2021/0369756 A1
(43) Pub. Date: Dec. 2, 2021

(54) USE OF CYCLIC ADENOSINE MONOPHOSPHATE, DERIVATIVE OR PRODRUG THEREOF IN PREPARATION OF DRUG FOR PREVENTING AND/OR TREATING DEPRESSION

(71) Applicant: CHINA PHARMACEUTICAL UNIVERSITY, Nanjing, Jiangsu (CN)

(72) Inventors: Guangji Wang, Nanjing, Jiangsu (CN); Jiye AA, Nanjing, Jiangsu (CN); Yue Zhang, Nanjing, Jiangsu (CN)

(73) Assignee: CHINA PHARMACEUTICAL UNIVERSITY, Nanjing, Jiangsu (CN)

(21) Appl. No.: 17/253,814

(22) PCT Filed: Aug. 31, 2020
(86) PCT No.: PCT/CN2020/112418

§ 371 Date: Dec. 18, 2020

(30) Foreign Application Priority Data
Sep. 17, 2019 (CN) 201910879011.4

(51) Int. Cl. A61K 31/7076; A61P 25/24

(52) U.S. Cl. CPC A61K 31/7076; A61P 25/24

(57) ABSTRACT

The disclosure discloses a use of cyclic adenosine monophosphate (cAMP), derivative or prodrug thereof in preparation of drug for preventing and/or treating depression. Using the established model of depressed mice caused by social defeat stress, cAMP is intraperitoneally injected upon constructing the model or directly injected into the nucleus accumbens brain region of depressed mice after modeling, which can significantly improve the depression-like behavior of mice. The disclosure discloses the prevention and treatment effect of cAMP on depression, and provides a candidate drug with new mechanism for clinical prevention and treatment of depression.

### CROSS REFERENCE TO RELATED APPLICATION

This application claims the priority of Chinese Patent Application No. 201910879011.4 entitled “Use of cyclic adenosine monophosphate, derivative or prodrug thereof in preparation of drug for treating depression” filed with China National Intellectual Property Administration on Sep. 17, 2019, which is incorporated herein by reference in its entirety.

### TECHNICAL FIELD

The disclosure relates to the use of drugs, especially relating to a use of cyclic adenosine monophosphate, derivative or prodrug thereof in preparation of drug for preventing and/or treating depression.

### BACKGROUND ART

About 350 million people worldwide suffer from depression, causing 800,000 suicides every year. Since 2017, depression has surpassed cancer and heart disease as the number one pusher for disability. However, the existing first-line drugs, monoamine reuptake inhibitors, are completely ineffective for at least one-third of depressive patients, and eventually result in more than half of recurrence, and even leading to side effects of suicide. Therefore, the development of truly effective antidepressant drugs is a clinical urgent problem to be solved.

Depression is a complex disease involving multiple mechanisms. It is generally considered to be related to genetic, social and environmental factors, but its specific pathogenesis is unclear. Clinical studies have shown that long-term pressure stress leads to immune dysregulation, for instance inflammation and depression are especially closely related, and proinflammatory cytokines in the circulatory system of depressed patients are significantly increased; at the same time, central microglia are activated and brain inflammation is aggravated. Moreover, studies have shown that there is a correlation between suicidal thoughts and brain inflammation in patients with depression. Animal studies have shown that simple peripheral inflammation is able to cause a series of depression-like behaviors, such as social avoidance, behavioral despair, deficits of sucrose preference, etc., and antidepressant drugs can significantly improve this phenomenon.

It is a question that how the peripheral inflammation causes depression in the brain. Previously, it was believed that pressure stress response increased the infiltration of peripheral monocytes into brain tissue, leading to excessive activation of inflammatory signals in the brain and abnormal synaptic function. However, with the application of double-gene edited mice (such as Ccr2RFP::Cx3cr1GFP mice) and visual imaging tools, etc., studies have found that the expression of cerebrovascular endothelial adhesion factors increases upon stress response, and a large number of monocytes are recruited to aggregate and release inflammatory factors. But due to the presence of the blood-brain barrier, peripheral monocytes cannot enter the brain parenchyma. The latest study shows that stress response leads to a significant decrease of the tight junction protein Cldn5 (a key component of the blood-brain barrier) in the nucleus accumbens brain region. Although peripheral monocytes cannot enter the brain parenchyma, the pro-inflammatory factor IL-6 (˜21.45 kDa) can enter the brain parenchyma in a large number. This phenomenon does not appear in the hippocampus and prefrontal cortex, which are the other key brain regions of depression, indicating that only a certain degree of damage occurs in the blood-brain barrier in the nucleus accumbens brain region. Nevertheless, it is not clear why there are specific pathological changes in the nucleus accumbens brain region and the molecular mechanism of this change.

### SUMMARY OF THE INVENTION

The object of the disclosure: The disclosure aims to provide a use of cyclic adenosine monophosphate (cAMP), derivative or prodrug thereof in preparation of drug for preventing and/or treating depression.

Technical Scheme: The disclosure provides a use of cyclic adenosine monophosphate, derivative or prodrug thereof in preparation of drug for preventing and/or treating depression. The structural formula of the cyclic adenosine monophosphate is as shown in formula I:

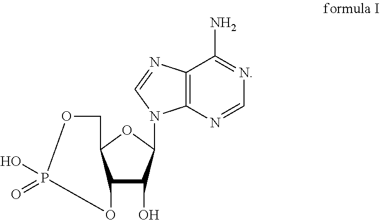

In some embodiments, the drug is a composition comprising cAMP as an active ingredient, pharmaceutically acceptable excipients and auxiliary ingredients.

In some embodiments, the dosage form of the drug includes tablets, capsules, patches, pastes, powders, oral liquids, suspensions, syrups, granules, dripping pills, orally disintegrating tablets and sustained-release tablets.

In some embodiments, the dosage form of the drug is an injection.

In some embodiments, the depression includes unipolar depression, bipolar depression, spirituality depression, reactive depression, secondary depression, seasonal depression, postpartum depression and menopausal depression.

In some embodiments, the prodrug is meglumine cyclic adenosine.

The disclosure also provides a use of cyclic adenosine monophosphate, derivative or prodrug thereof for preventing and/or treating depression.

In some embodiments, an administration method for preventing and/or treating depression includes injection administration and oral administration.

The brain region tied to depression is studied in detail by using metabonomics methods, and it is found that the level of cyclic adenosine monophosphate (cAMP) in the nucleus accumbens brain region is significantly decreased in the depression sensitive group compared with hippocampus and prefrontal cortex, and it is significantly correlated to the severity of depression. Meglumine cyclic adenosine is the meglumine salt form of cyclic adenosine monophosphate to increase the transmembrane ability of cyclic adenosine monophosphate. By giving mice meglumine cyclic adenosine to supplement cAMP exogenously, the destruction of the blood-brain barrier in the nucleus accumbens brain region of mice may be reversed, and depression-like behavior in mice may be significantly improved.

Beneficial effect: the disclosure evaluates the antidepressant effect of cAMP given by intraperitoneal injection and intracerebral nucleus accumbens injection through establishing chronic social defeat stress (CSDS) model for C57BL/6J mice. Studies have found that intraperitoneal injection to supplement cAMP can reduce the incidence of depression and sensitivity after social frustration, significantly improve the blood-brain barrier damage, and reduce the immobility time of tail suspension and immobility time in forced swimming test. A single injection to supplement cAMP into nucleus accumbens in the brain can gradually improve the social avoidance behavior of mice within a week, and reduce the immobility time of tail suspension and immobility time in forced swimming test. It is suggested that supplementation of cAMP has the effect of preventing and treating depression, which is expected to be used in the treatment of clinical depression after research.

### BRIEF DESCRIPTION OF THE DRAWINGS

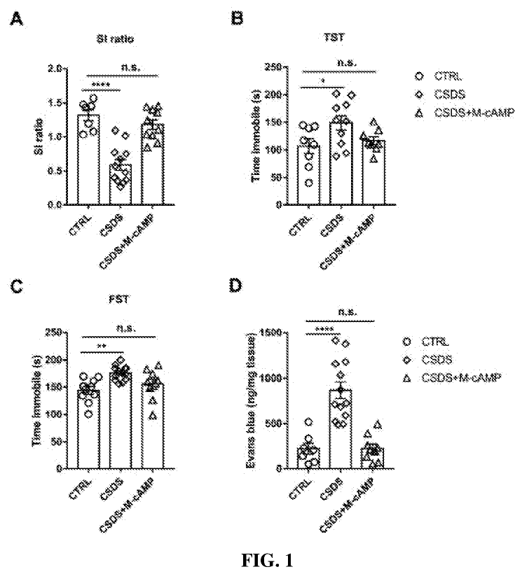

FIG. 1 shows the antidepressant effect of intraperitoneal injection of cAMP, wherein,
A: A statistical graph of the social coefficients of mice in each group: CTRL represents the normal control group, CSDS represents the social defeat model group, and CSDS+M-cAMP represents the social defeat model combined with the M-cAMP administration group, using one-way ANOVA analysis, ****p<0.0001 vs CTRL, n.s. represents no significant difference;
B: A statistical graph of immobility time of tail suspension of mice in each group;
C: A statistical graph of immobility time in forced swimming of mice in each group;
D: A statistical graph of Evans Blue fluorescence intensity in brain parenchyma of nucleus accumbens of mice in each group,
wherein, CTRL represents the normal control group, CSDS represents the social defeat model group, and CSDS+M-cAMP represents the social defeat model combined with the M-cAMP administration group, using one-way ANOVA analysis, *p<0.05, **p<0.01, ****p<0.0001 vs CTRL, n.s. represents no significant difference;

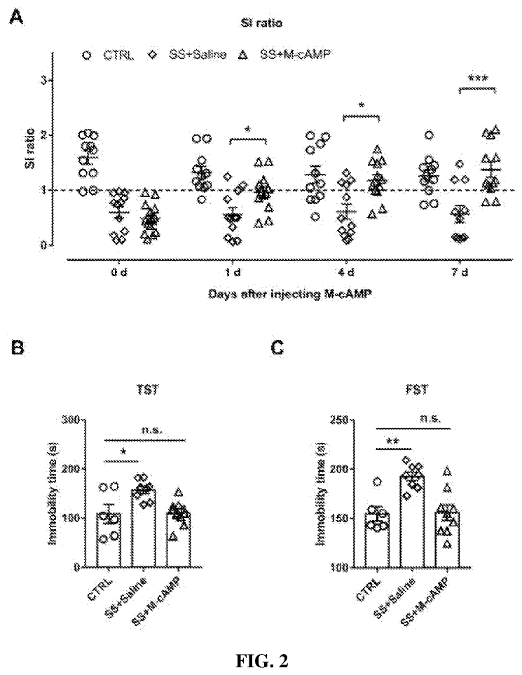

FIG. 2 shows the antidepressant effect of nucleus accumbens brain region injection of cAMP, wherein,
A: A statistical graph of the social coefficient of mice in each group at different times after injection;
B: A statistical graph of immobility time of tail suspension of mice in each group;
C: A statistics of immobility time in forced swimming of mice in each group;
wherein, CTRL represents the normal control group, SS represents the depression sensitive group, SS+M-cAMP represents the depression sensitive group combined with nucleus accumbens administration group of M-cAMP, using one-way ANOVA analysis, *p<0.05, **p<0.01,***P<0.001, n.s. represents no significant difference.

### DETAILED DESCRIPTION OF THE EMBODIMENTS

### Example 1 the Antidepressant Effect of Intraperitoneal Injection of Meglumine Cyclic Adenosine

CD-1 mice (retired mice, male, 4˜6 months old, purchased from Beijing Vital River Laboratory Animal Technology Co., Ltd., weight 48±5 g) and C57BL/6J mice (male, 7˜8 weeks, purchased from Beijing Vital River Laboratory Animal Technology Co., Ltd., weight 20±2 g) were used in chronic social defeat stress model, and were fed adaptively for one week under a standard feeding environment (free diet and drinking, under 12-h light/12-h dark cycles.).

Establishment of animal model: CD-1 mice were successively screened for 3 days, and aggressive CD-1 mice were screened for modeling. CD-1 mice and C57 mice were separated with a perforated transparent plastic separator. The C57 mice were placed on the same side of CD-1 mice every day to receive attack for 10 minutes each time, and the CD-1 mouse attacker was changed every day. The C57 mice were then placed on the opposite side after being attacked, and were continuously received visual and gustatory stress from the CD-1 mice in the rest of 24 hours. The operation was repeated for 10 consecutive days.

Methods of administration: During the establishment of the social defeat stress model, M-cAMP (meglumine cyclic adenosine, 10 mg/kg) was injected intraperitoneally 2 hours before the mice were placed in the model condition every day to supplement cAMP in vivo, an equal volume of normal saline was given to the control group.

Evaluation and grouping of the model are as follows: After 10 days of modeling, depression sensitivity in mice was detected by the social avoidance test. That is, in an open area, the movement trajectory of C57 mice was recorded for 150 s in the absence and in the presence of CD-1 mice, and the time was calculated once C57 mice entered the social area. The ratio of the time that C57 mice enter the social area in the presence of CD-1 mice to the time that C57 mice enter the social area in the absence of CD-1 mice is used as the social coefficient. If the social coefficient is less than 1, it is judged as a depression sensitive group, if the social coefficient is greater than 1, it is judged as a depression tolerance group. The results show that intraperitoneal injection of M-cAMP to supplement cAMP in vivo can reduce the proportion of depression sensitivity (FIG. 1 (A)).

Effect on depression-like behavior in mice: Tail suspension test and forced swimming test were used to evaluate depression-like behavior. In the tail suspension test, the tails of the mice (about 2.5 cm from the tip of tail) were taped to a scaffold, and the heads of the mice were about 30 cm away from the ground. At the same time, the activity state of the mice was recorded on video for 6 minutes. The immobility time in the next 4 minutes was calculated by two experimenters in a single-blind condition. The results show that intraperitoneal injection of M-cAMP to supplement cAMP in vivo can significantly reduce the immobility time of C57 mice in the tail suspension test (FIG. 1 (B)). In the forced swimming test, C57 mice were carefully placed in a 5 L glass beaker (the water depth in the cup was about 20 cm), and the water temperature was 24±1° C. The activity state of the mice was recorded on video, and the test lasted for 6 minutes. The immobility time in the next 4 minutes was calculated by two experimenters in a single-blind condition after the test. The judging standard for the immobility state is that the mouse stops struggling or appears to be floating, and the limbs are immobile or only small moving to float the head on the water surface. A new beaker of water was changed for different mice. The results show that intraperitoneal injection to supplement cAMP can significantly reduce the immobility time of C57 mice in the forced swimming test (FIG. 1 (C)).

Effects on the permeability of the blood-brain barrier in the nucleus accumbens brain region: C57 mice were injected with 2% Evans Blue (0.12 mL/20 g) through the tail vein, the heart was perfused after circulating for 16 hours, and the nucleus accumbens was homogenized and extracted to detect the fluorescence intensity of Evans Blue (Ex=633 nm; Em=670 nm). The results show that intraperitoneal injection of M-cAMP to supplement cAMP in vivo can significantly inhibit the leakage of Evans Blue into the brain parenchyma of nucleus accumbens brain region (FIG. 1 (D)).

### Example 2 the Antidepressant Effect of Intracerebral Nucleus Accumbens Injection of Meglumine Cyclic Adenosine

The establishment and grouping of experimental animals and models were under the same condition as Example 1. After modeling, the depression-sensitive mice were screened according to the social coefficient and randomly divided into a normal saline group and an intracerebral nucleus accumbens injection of M-cAMP group. Mice were carefully and correctly fixed on the mouse stereotaxic apparatus after the mice were anesthetized. The skin of the head was disinfected, then the skull was carefully exposed, the bregma was positioned as the zero point, and 0.5 μL of M-cAMP or normal saline at 0.1 μL/min was separately injected into the bilateral nucleus accumbens brain region according to the stereotactic coordinates of mouse brain (1.6 mm in front, 1.4 mm on side, 4.4 mm deep). After the injection, the microinjector was stayed in place for 10 minutes and then slowly removed, thereby preventing liquid overflow. The skull was sealed with an appropriate amount of bone wax, the skin of head was sutured, and the mice were observed and fed. The social avoidance behavior of the mice was evaluated on days 1, 4, and 7 after the administration. The depression-like behavior of the mice was evaluated on days 8 and 9, respectively, using the tail suspension test and the forced swimming test.

The results show that intracerebral nucleus accumbens injection of M-cAMP to supplement cAMP in vivo can significantly improve the social avoidance behavior of mice within a week. The social coefficient of mice after injected M-cAMP is significantly different from the normal saline group (FIG. 2 (A)). In the tail suspension test, the immobility time of mice is significantly reduced after intracerebral nucleus accumbens injection of M-cAMP (FIG. 2 (B)), and in the forced swimming test, the immobility time of mice is significantly reduced after administration of M-cAMP (FIG. 2 (C)).

The above described are only preferred embodiments of the disclosure, it should be understood by those skilled in the art that, without departing from the principle of the disclosure, several improvements and modifications can be made, and these improvements and modifications also should be regarded as the protection scope of the disclosure fall into the scope of the disclosure.

1. A method of preventing and/or treating depression comprising administering to a subject in need thereof a composition comprising cyclic adenosine monophosphate (cAMP), a derivative of cAMP, or a prodrug of cAMP, wherein the structural formula of the cAMP is as shown in formula I:

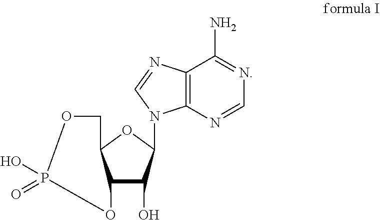

2. The method according to claim 1, wherein the composition further comprises a pharmaceutically acceptable excipient and an auxiliary ingredient.

3. The method according to claim 2, wherein the dosage form of the composition is selected from the group consisting of: a tablet, a capsule, a patch, a paste, a powder, an oral liquid, a suspension, a syrup, a granule, a dripping pill, an orally disintegrating tablet, and a sustained-release tablet.

4. The method according to claim 2, wherein the dosage form of the composition is an injection.

5. The method according to claim 1, wherein the depression is selected from the group consisting of: unipolar depression, bipolar depression, spirituality depression, reactive depression, secondary depression, seasonal depression, postpartum depression and menopausal depression.

6. The method according to claim 1, wherein the prodrug is meglumine cyclic adenosine (M-cAMP).

7. (canceled)

8. The method according to claim 1, wherein the composition is administered orally or via injection.
